(19) United States
(12) Patent Application Publication
(10) Pub. No.: US 2022/0119028 A1
(43) Pub. Date: Apr. 21, 2022

(54) Shopping Cart Handle Cover with Integrated Dispenser

(71) Applicants: Tamara Hopkins, Lancaster, CA (US); Na'Hsagui Hopkins James, Lancaster, CA (US)

(72) Inventors: Tamara Hopkins, Lancaster, CA (US); Na'Hsagui Hopkins James, Lancaster, CA (US)

(21) Appl. No.: 17/505,951

(22) Filed: Oct. 20, 2021

Related U.S. Application Data
(60) Provisional application No. 63/094,278, filed on Oct. 20, 2020.

(51) Int. Cl. B62B 5/06; A61L 2/18; B62B 3/14

(52) U.S. Cl. CPC B62B 5/069; A61L 2/18; B08B 1/006; A61L 2202/16; A61L 2202/17; B62B 3/1472

(57) ABSTRACT

A shopping cart handle cover with integrated dispenser is provided. The device includes an elongated body having a first end opposite a second end. A slot is disposed on a lower side of the elongated body extending along a longitudinal axis thereof, wherein the slot is dimensioned to removably secure a shopping cart handle therein. An internal reservoir is disposed within the elongated body, wherein the internal reservoir is in fluid communication with a dispensing aperture disposed through an upper side of the elongated body. The internal reservoir is deformable to force a fluid stored therein through the dispensing aperture when the elongated body is compressed. In some embodiments, one or more accessory containers affixed to an exterior of the elongated body.

### CROSS REFERENCE TO RELATED APPLICATIONS

This application claims the benefit of U.S. Provisional Application No. 63/094,278 filed on Oct. 20, 2020. The above identified patent application is herein incorporated by reference in its entirety to provide continuity of disclosure.

### BACKGROUND OF THE INVENTION

The present invention relates to shopping cart sanitization systems. More particularly, the present invention pertains to a shopping cart handle cover with an integrated dispenser for dispensing sanitizing fluid therefrom for cleansing a user's hands and the shopping cart handle.

Many individuals utilize public shopping carts on a regular basis, however, such publicly utilized objects can be a primary vector for transmission of germs, viruses, bacteria, or other pathogens. If transmitted, these pathogens may cause severe illnesses which may lead to hospitalization and even death. Typically, shopping carts are not frequently sanitized between individual uses, which can leave pathogens on the shopping cart handle. When a subsequent shopper uses the cart, they may grasp the handle and then touch their face and mouth, thereby transferring pathogens into their system.

In order to inhibit the spread of pathogens, many individuals use hand sanitizer or disinfectant wipes to clean their hands or surfaces to eliminate germs and pathogens. However, individuals may not consider bringing hand sanitizer on their shopping outings, and as such be unable to properly disinfect their hands after touching public surfaces. Similarly, disinfectant wipes may not be readily available to wipe down shopping cart handles or other public surfaces between uses. Alternatively, individuals may bring a cloth or other barrier place over the shopping cart, handle during use, however such covers are often merely resting on the shopping cart handle and are prone to failing to the ground and becoming contaminated. Therefore, a device that can provide a securable barrier between the shopping cart handles and the user while also dispensing a sanitizing fluid is desired.

In light of the devices disclosed in the known art, it is submitted that the present invention substantially diverges in design elements from the known art and consequently It is clear that there is a need in the art for an improvement to existing shopping cart sanitization systems. In this regard, the instant invention substantially fulfills these needs.

### SUMMARY OF THE INVENTION

In view of the foregoing disadvantages inherent in the known types of shopping cart, sanitization systems now present in the known art, the present invention provides a shopping cart handle cover wherein the same can be utilized for providing convenience for the user when dispensing hand sanitizer or other sanitizing fluid for disinfecting the shopping cart handle or a user's hands.

The present system comprises an elongated body having a first end opposite a second end. A slot is disposed on a lower side of the elongated body extending along a longitudinal axis thereof, wherein the slot is dimensioned to removably secure a shopping cart handle therein. An internal reservoir is disposed within the elongated body, wherein the internal reservoir is in fluid communication with a dispensing aperture disposed through an upper side of the elongated body. The internal reservoir is deformable to force a fluid stored therein through the dispensing aperture when the elongated body is compressed. In some embodiments, one or more accessory containers is affixed to an exterior of the elongated body,

In some embodiments, the slot extends along an entire length of the elongated body, such that the slot extends through the first end and the second end. In another embodiment, the slot comprises a cylindrical cross section. In other embodiments, a lower end of the slot comprises a lengthwise opening having a width less than a width of the slot, such that the lower end biases about the shopping cart handle to removably secure the elongated body thereto via frictional engagement. In yet another embodiment, the dispensing aperture is disposed adjacent to the first end of the elongated body. In some embodiments, the dispensing aperture comprises an elongated opening parallel to the longitudinal axis of the elongated body. In another embodiment, the internal reservoir terminates at a distal end of the dispensing aperture. In other embodiments, the internal reservoir mimics the contours of the exterior of the elongated body. In yet another embodiment, a refilling opening in fluid communication with the internal reservoir disposed through the elongated body. In some embodiments, the one or more accessory containers are removably securable to the elongated body via complementary fasteners. In another embodiment, the complementary fasteners comprise hook and loop fasteners.

### BRIEF DESCRIPTION OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a perspective view of an embodiment of the shopping cart handle cover with integrated dispenser.

FIG. 2 shows a close-up view of the slot of an embodiment of the shopping cart handle cover with integrated dispenser.

FIG. 3 shows a cross-sectional view of an embodiment of the shopping cart handle cover with integrated dispenser.

FIG. 4 shows a perspective view of an embodiment of the shopping cart handle cover in use.

### DETAILED DESCRIPTION OF THE INVENTION

Reference is made herein to the attached drawings. Like reference numerals are used throughout the drawings to depict like or similar elements of the shopping cart handle cover with integrated dispenser. The figures are intended for representative purposes only and should not be considered to be limiting in any respect.

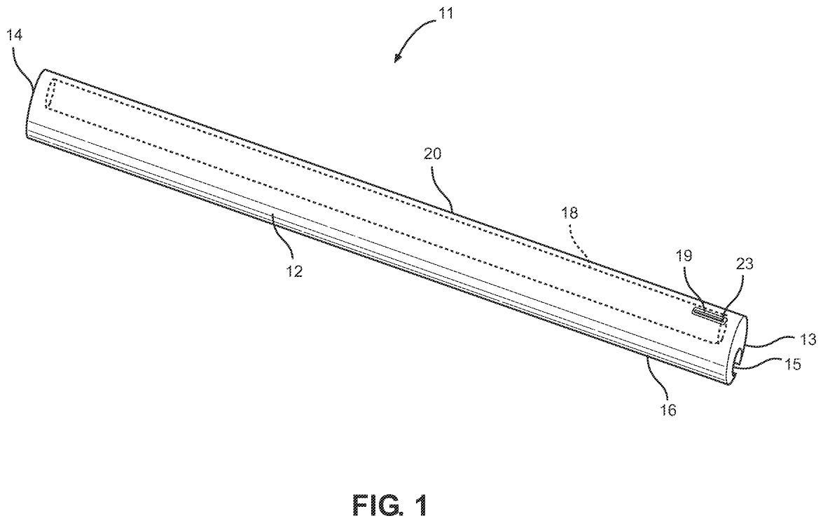

Referring now to FIG. 1, there is shown a perspective view of an embodiment of the shopping cart handle cover with integrated dispenser. The shopping cart handle cover with integrated dispenser 11 comprises an elongated body 12 having a first end 13 opposite a second end 14. An internal reservoir 18 is disposed within the elongated body 12, wherein the internal reservoir 18 is in fluid communication with a dispensing aperture 19 disposed on an upper side 20 of the elongated body 12. The internal reservoir 18 is configured to retain a fluid therein, such as disinfectant or hand sanitizer, whereupon pressure being applied to the elongated body 12, the fluid is driven through the internal reservoir 18 to exit via the dispensing aperture 19. In some embodiments, the elongated body 12 comprises a flexible and deformable material, such as neoprene, such that the elongated body 12 provides a cushioned surface that is capable of deformation to dispense a fluid stored therein. In the illustrated embodiment, the internal reservoir 18 extends along a longitudinal axis of the elongated body 12, such that a majority of the elongated body 12 can be depressed to dispense fluid from the internal reservoir 18. Alternatively, in some embodiments, the internal reservoir 18 is disposed along 3 portion of the elongated body 12 adjacent to the dispensing aperture 19, such that utilization of the shopping cart handle cover with integrated dispenser 11 as a sanitary gripping surface for a shopping cart does not inadvertently dispense fluid from the internal reservoir 18. In the shown embodiment, the internal reservoir 18 terminates at a distal end 23 of the dispensing aperture 19, such that pressure need only be applied on a single side of the dispensing aperture 19 to dispense fluid therefrom.

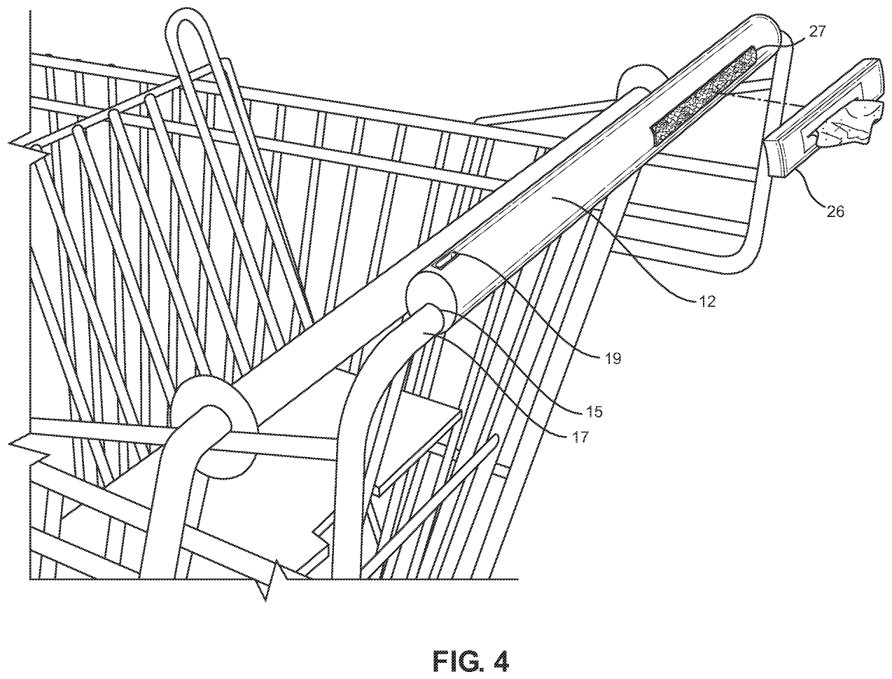

A slot 15 extends through the elongated body 12 on a lower side 16 thereof, wherein the slot 15 is dimensioned to removably secure the elongated body 12 to a shopping cart handle (as shown in FIG. 4, 17). In the shown embodiment, the slot. 15 extends along an entirety of the elongated body 12, such that the slot 15 extends through each of the first and second ends 13,14 of the elongated body 12. In this manner, the elongated body 12 can be removably secured to a shopping cart handle having a length greater than that of the shopping cart handle cover with integrated dispenser 11. In the shown embodiment, the slot 15 extends parallel to the longitudinal axis of the elongated body 12, such that the slot 15 is disposed parallel to the internal reservoir 18. In the illustrated embodiment, the slot 15 comprises a cylindrical cross-section, however, in alternate embodiments, the slot 15 is dimensioned to conform to alternate shapes of typical shopping cart, handles. In some such embodiments, the slot 15 comprises a smaller cross-sectional area than an associated shopping cart handle, such that the slot 15 frictionally engages the shopping cart handle to ensure the shopping cart handle cover 11 is secured thereto.

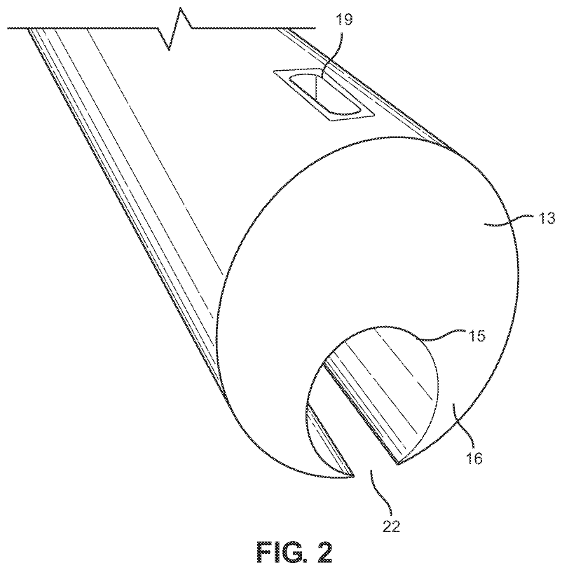

Referring now to FIG. 2., there is shown a close-up view of the slot of an embodiment of the shopping cart handle cover with integrated dispenser. In the shown embodiment, the slot 15 extends through the first end 13 of the elongated body, such that a shopping cart handle of greater length than that of the elongated body can be secured therein. Furthermore, in the illustrated embodiment, the slot 15 comprises a lengthwise opening 22 disposed along a lower end of the slot 15, wherein the lengthwise opening 22 comprises a smaller diameter than a diameter of the slot 15. In this manner, the slot 15 is configured to bias outwardly to receive the shopping cart handle through the lengthwise opening 22, whereupon the lengthwise opening 22 biases inwardly to frictionally engage the shopping cart handle within the slot 15. As such, the elongated body can be removably secured to shopping cart handles of various diameters, as the slot 15 is configured to wrap about the shopping carl handles to increase frictional engagement therewith. In some embodiments, an interior surface of the slot 15 comprises a high friction material to increase frictional engagement with the shopping cart handle. In the shown embodiment, the dispensing aperture 19 is disposed adjacent to the first end 13 and parallel to the length of the slot 15 to facilitate dispensing of the fluid within the internal reservoir pressure applied on one side of the dispensing aperture 19.

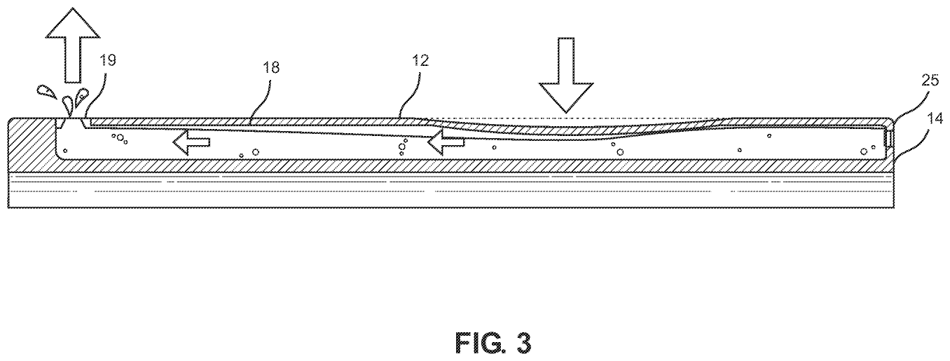

Referring now to FIG. 3, there is shown a cross-sectional view of an embodiment of the shopping cart handle cover with integrated dispenser. In the illustrated embodiment, the elongated body 12 is shown being depressed, which causes the internal reservoir 18 to deform, thereby forcing fluid disposed within the internal reservoir 18 to exit through the dispensing aperture 19. In some embodiments, the internal reservoir 18 conforms to the dimensions of the elongated body 12, such that the volume of the internal reservoir 18 is maximized. Furthermore, this reduces the thickness of the sidewall of the elongated body 12, facilitating deformation of the elongated body 12 to dispense fluid therefrom. In some embodiments, the fluid is disposed directly within the internal reservoir 18, however, in alternate embodiments, a separate internal bladder is disposed within the internal reservoir 18, wherein the internal bladder is similarly deformable to direct fluid therein through the dispensing aperture 19. In this manner, the internal bladder may be removable from the internal reservoir 18, such that additional fluid can be placed within the internal bladder or to replace a damaged bladder. Alternatively, in the shown embodiment, a separate refilling aperture 25 is disposed through the elongated body 12 on the second end 14, wherein the refilling aperture 25 is in fluid communication with the internal reservoir 18. The refilling aperture 25 is contemplated to be disposed on an end of the elongated body 12 opposite the dispensing aperture 19, such that the fluid within the internal reservoir 18 is directed primarily to the dispensing aperture 19. In some embodiments, the refilling aperture 25 further comprises a one-way valve thereon, such as a flap valve, to prevent, fluid from exiting the refilling aperture 25 when pressure is applied to an exterior of the elongated body 12. Furthermore, in the shown embodiment, an end wall of the internal reservoir 18 disposed adjacent to the dispensing aperture 19 comprises an arcuate shape, such that fluid is guided toward the dispensing aperture 19 along the arcuate end wall of the internal reservoir 18.

Referring now to FIG. 4, there is shown a perspective view of an embodiment of the shopping cart handle cover in use. In the illustrated embodiment, the shopping cart handle cover further comprises an accessory container 26 affixed to an exterior of the elongated body 12, wherein the accessory container 26 can store additional accessories that may be required during 3 typical shopping trip. In the shown embodiment, the accessory container 26 comprises a disinfectant wipe pouch to provide additional sanitization means to the user. In some embodiments, the accessory container 26 is affixed to the elongated body 12 along a portion of the elongated body 12 through which the internal reservoir does not extend, such that interaction with the accessory container 26 does not deform the elongated body 12 to dispense fluid from the dispensing aperture 19. Furthermore, in the illustrated embodiment, the accessory container 26 is removably securable to the elongated body 12 via complementary fasteners 27 disposed on a rear side of the accessory container 26 and an exterior of the elongated body 12. In this manner, the user can remove a depleted accessory container 26, or utilize additional interchangeable accessory containers 26 storing alternate items for use when shopping.

In one use, the elongated body 12 is removably secured to a shopping cart handle 17 via the slot 15, such that the slot 15 frictionally engages the shopping cart handle 17. The elongated body 12 can be affixed to an existing shopping cart handle 17 by the shopping establishment, or alternatively, be affixed by the user to an existing shopping cart at the start of a shopping trip. Once affixed, the elongated body 12 can then be depressed via external pressure to drive sanitizing fluid, such as disinfectant or hand sanitizer through the dispensing aperture 19. Once the fluid is dispensed, the user can wipe down the length of the elongated body 12 to disinfect the grasping surface of the shopping cart handle 17. Additionally, the dispensed fluid can be utilized to sanitize other items In the vicinity to minimize the spread of pathogens, in this manner, the user can be assured a safe and germ free shopping experience.

It is therefore submitted that the instant invention has been shown and described in various embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly, and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A shopping cart handle cover with integrated dispenser, comprising:
an elongated body having a first end opposite a second end;
a slot disposed on a lower side of the elongated body extending along a longitudinal axis thereof;
wherein the slot is dimensioned to removably secure a shopping cart handle therein;
an internal reservoir disposed within the elongated body;
wherein the internal reservoir is in fluid communication with a dispensing aperture disposed through an upper side of the elongated body;
wherein the internal reservoir is deformable to force a fluid stored therein through the dispensing aperture when the elongated body is compressed.

2. The shopping cart handle cover of claim 1, wherein the slot extends along an entire length of the elongated body, such that the slot extends through the first end and the second end.

3. The shopping cart handle cover of claim 1, wherein the slot comprises a cylindrical cross section.

4. The shopping cart handle cover of claim 1, wherein a lower end of the slot comprises a lengthwise opening having a width less than a width of the slot, such that the lower end biases about the shopping cart handle to removably secure the elongated body thereto via frictional engagement.

5. The shopping cart handle cover of claim 1, wherein the dispensing aperture is disposed adjacent to the first end of the elongated body.

6. The shopping cart handle cover of claim 1, wherein the dispensing aperture comprises an elongated opening parallel to the longitudinal axis of the elongated body.

7. The shopping cart handle cover of claim 1, wherein the internal reservoir terminates at a distal end of the dispensing aperture.

8. The shopping cart handle cover of claim 1, wherein the internal reservoir mimics the contours of an exterior of the elongated body.

9. The shopping cart handle cover of claim 1, further comprising a refilling opening in fluid communication with the internal reservoir disposed through the elongated body.

10. A shopping cart handle cover with integrated dispenser, comprising:
an elongated body having a first end opposite a second end;
a slot disposed on a lower side of the elongated body extending along a longitudinal axis thereof;
wherein the slot is dimensioned to removably secure a shopping cart handle therein;
an internal reservoir disposed within the elongated body;
wherein the internal reservoir is in fluid communication with a dispensing aperture disposed through an upper side of the elongated body;
wherein the internal reservoir is deformable to force a fluid stored therein through the dispensing aperture when the elongated body is compressed;
one or more accessory containers affixed to an exterior of the elongated body.

11. The shopping cart, handle cover of claim 10, wherein the slot extends along an entire length of the elongated body, such that the slot extends through the first end and the second end.

12. The shopping cart handle cover of claim 10, wherein the slot comprises a cylindrical cross section.

13. The shopping cart handle cover of claim 10, wherein a lower end of the slot comprises a lengthwise opening having a width less than a width of the slot, such that the lower end biases about the shopping cart handle to removably secure the elongated body thereto via frictional engagement.

14. The shopping cart handle cover of claim 10, wherein the dispensing aperture is disposed adjacent to the first end of the elongated body.

15. The shopping cart handle cover of claim 10, wherein the dispensing aperture comprises an elongated opening parallel to the longitudinal axis of the elongated body.

16. The shopping cart handle coyer of claim 10, wherein the internal reservoir terminates a distal end of the dispensing aperture.

17. The shopping cart handle cover of claim 10, wherein the internal reservoir mimics the contours of the exterior of the elongated body.

18. The shopping cart handle cover of claim 10, further comprising a refilling opening in fluid communication with the internal reservoir disposed through the elongated body.

19. The shopping cart handle cover of claim 10, wherein the one or more accessory containers are removably securable to the elongated body via complementary fasteners.

20. The shopping cart handle cover of claim 19, wherein the complementary fasteners comprise hook and loop fasteners.
